(19) United States
(12) Patent Application Publication
(10) Pub. No.: US 2023/0191146 A1
(43) Pub. Date: Jun. 22, 2023

(54) Therapeutic device for providing infrared radiation treatment

(71) Applicant: Visage Sculpture, S-corp., Newton, MA (US)

(72) Inventor: Maria Amelina, Newton, MA (US)

(21) Appl. No.: 18/078,974

(22) Filed: Dec. 11, 2022

Related U.S. Application Data
(60) Provisional application No. 63/291,442, filed on Dec. 19, 2021.
(60) Provisional application No. 63/298,261, filed on Jan. 11, 2022.

(51) Int. Cl. A61N 5/06; A61N 5/067

(52) U.S. Cl. CPC A61N 5/0616; A61N 5/067; A61N 5/0625; A61N 2005/0645; A61N 2005/0659; A61N 2005/0652; A61N 2005/0647

(57) ABSTRACT

A device for therapeutic skin treatment using infrared radiation is provided in accordance with the embodiments of the present invention. It is portable and includes portable rechargeable batteries, such that it can be used on the go (while driving, for example). Furthermore, it is safe and designed to resemble everyday products to facilitate inconspicuous treatments so nobody will know a device is being used by a user for rejuvenating purposes, which attends to the user's privacy considerations. Moreover, the device of the present invention is inexpensive, easy to operate and effective and efficient in providing infrared radiation-based skin rejuvenating treatments, incorporating infrared radiation elements positioned in close proximity to the skin.

### CROSS-REFERENCE TO RELATED APPLICATIONS

This application claims the benefit of U.S. provisional patent application No. 63/291,442 filed on Dec. 19, 2021 and U.S. provisional patent application No. 63/298,261 filed on Jan. 11, 2022, the contents of which are incorporated herein by reference in their entirety.

### TECHNICAL FIELD

Embodiments disclosed herein relate generally to therapeutic devices for providing skin treatment, and more particularly to therapeutic devices for providing infrared radiation-based skin treatments.

### BACKGROUND ART

Phototherapy is well known and practiced for a very long time. For instance, short wavelengths light was used to treat skin infection, such as lupus vulgaris; and red light was used to treat smallpox lesions. Today, light is used to treat various diseases and conditions, including pain management, hair growth, and skin conditions (e.g., psoriasis, acne, acne vulgaris, eczema, neonatal jaundice, atopic dermatitis, etc.). For each therapy, it is costumed to use a different type of light, e.g., laser, red light, near infrared and ultraviolet lights, etc. Certain phototherapy treatments can be performed at home, but others, which uses high-power or harmful rays (e.g., UV) are typically performed by well trained professionals, which expose only the treated skin area to the rays and avoid exposure of healthy skin to such rays.

Infrared (IR) radiation is an invisible portion of the electromagnetic spectrum adjacent to the long wavelength of the visible light range and extends to the microwave range. IR radiation consists of wavelength ranging from 0.75 to 1000 μm (0.75 μm=750 nm), and can be subdivided into near (0.75 to 3 μm), middle (3 to 30 μm), and far (30 to 1000 μm).

Effects of infrared radiation on skin photo-aging and pigmentation are known. It has been shown that IR radiation treatment at an ambient temperature is safe and does not cause harmful thermal injuries. Furthermore, IR radiation may result in beneficial effect on skin texture and wrinkles by increasing collagen and elastin in the dermis through stimulation of fibroblasts.

However, the prior art devices utilizing light and magnetic energies in general and infrared radiation in particular suffer from significant structural and functional shortcomings. These devices are bulky, not portable, difficult to operate, expensive, and complicated to assemble, as well as not designed for stealth treatments (i.e., the use of devices resembling everyday products to facilitate inconspicuous treatments). Additionally, these prior art devices are not effective and efficient in providing rejuvenating treatments because they employ heating elements that are not in close proximity to the skin of a user.

Therefore, there is a need for a therapeutic device that overcomes the mentioned above shortcomings of the known prior art devices.

### Summary of the Embodiments

A device for therapeutic skin treatment using infrared radiation is provided in accordance with the embodiments of the present invention. It is portable and includes portable rechargeable batteries, such that it can be used on the go (while driving, for example). Furthermore, it is safe and designed to resemble everyday products to facilitate inconspicuous treatments so nobody will know a device is being used by a user for rejuvenating purposes, which attends to the user's privacy considerations. Moreover, the device of the present invention is inexpensive, easy to operate and effective and efficient in providing infrared radiation-based skin rejuvenating treatments, incorporating infrared radiation elements positioned in close proximity to the skin. Even more importantly, the devices of the present invention are configured to be used not only to treat skin but also to prevent diseases, including Covid-19 infections, emitting infrared radiation with the wavelength of 5 microns to 20 microns, in which range the human immune system is significantly enhanced.

According to one embodiment of the present invention, the therapeutic skin treatment device includes a mask with an electrically operated infrared radiation element embedded into the mask and electrically connected to a battery by wiring. The infrared radiation element can be one or more strips with a plurality of infrared LEDs attached thereon. In some instances, it can be one or more infrared lasers, and yet in some other preferred instances, it can be one or more heating pads embedded into the mask.

The infrared radiation element is configured to emit infrared radiation in the middle infrared range of 3-30 μm. In some instances, the infrared radiation element is configured to emit radiation in the range of 30-100 μm. Preferably, in the range of 6-10 μm, and most preferably at 8.5 μm. The face mask can be used alone or in conjunction with the gloves, as a kit for skin rejuvenating treatment of both hands and a face.

According to another aspect of the invention, the therapeutic skin treatment device can include a pair of gloves with an eclectically operated infrared radiation element embedded into the gloves. The infrared radiation element can be one or more strips with a plurality of infrared LEDs attached thereon. In some instances, it can be one or more infrared lasers, and yet in some other preferred instances, it can be one or more heating pads embedded into the gloves.

Other aspects, embodiments and features of the device and method will become apparent from the following detailed description when considered in conjunction with the accompanying figures. The accompanying figures are for schematic purposes and are not intended to be drawn to scale. In the figures, each identical or substantially similar component that is illustrated in various figures is represented by a single numeral or notation. For purposes of clarity, not every component is labeled in every figure. Nor is every component of each embodiment of the device and method shown where illustration is not necessary to allow those of ordinary skill in the art to understand the device and method.

### BRIEF DESCRIPTION OF THE DRAWINGS

The preceding summary, as well as the following detailed description of the disclosed system and method, will be better understood when read in conjunction with the attached drawings. It should be understood, however, that neither the device nor the method is limited to the precise arrangements and instrumentalities shown.

FIG. 1 is a perspective view of a disclosed therapeutic device embodying the invention for facial rejuvenation treatment.

FIG. 2 is a perspective view of the device in accordance with an alternative wiring and battery configuration.

FIG. 3 is a perspective view showing another embodiment of the disclosed device.

FIG. 4 is a perspective view of a disclosed therapeutic device embodying the invention for rejuvenating treatment of hands.

FIG. 5 is a perspective view of the device of FIG. 4 showing the wiring arrangement.

FIG. 6 is a perspective view of the device of FIG. 4 illustrating infrared light heating pads embedded into the gloves.

FIG. 7A and FIG. 7B illustrate a comparison of the skin condition before and after the treatment using the disclosed device.

FIG. 8 shows the disclosed device incorporating the infrared radiation element into the face mask in a pattern according to an embodiment of the present invention.

FIG. 9 shows the disclosed device incorporating the infrared radiation element into the gloves in a pattern according to an embodiment of the present invention.

### DETAILED DESCRIPTION OF SPECIFIC EMBODIMENTS

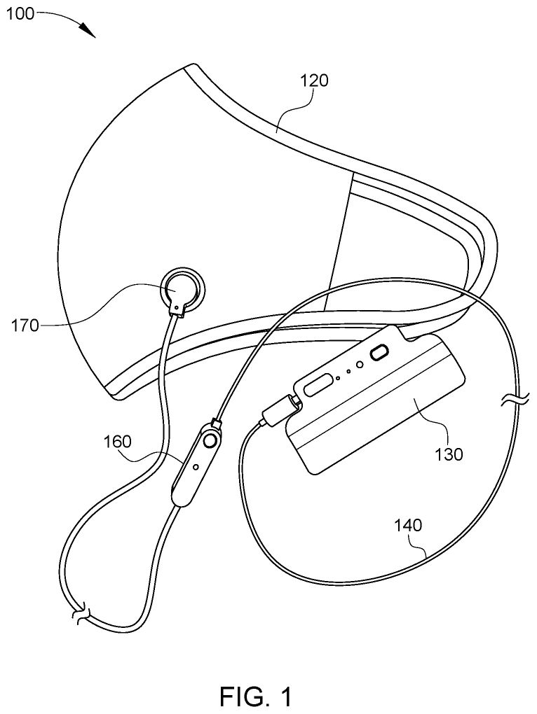

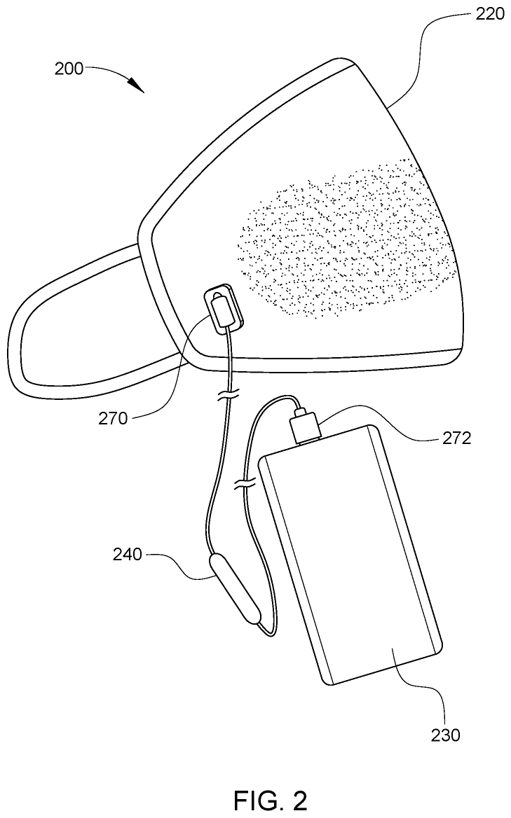

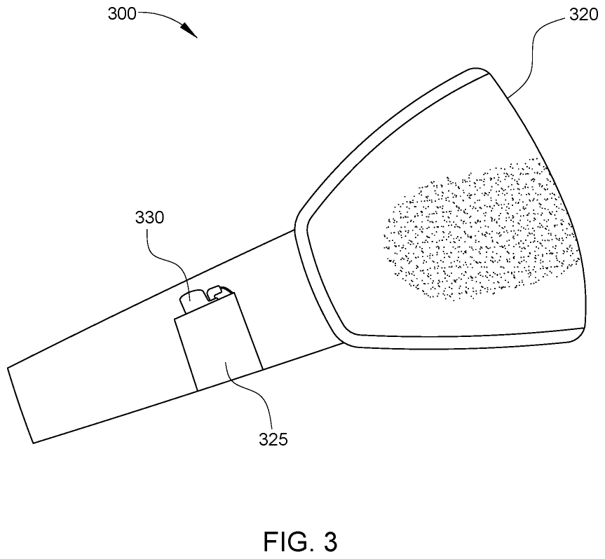

FIG. 1 shows therapeutic device 100 for providing infrared radiation treatment of skin. It includes face mask 120, an infrared radiation element embedded into the face mask 120 (not shown), portable battery 130 connected by electrical wire 140 to plug 170 and power button 160. The battery 130 can preferably be a Li-ion battery having the battery voltage of 7.4V, capacity of 2200 mAh, charging voltage peak of 8.4±0.05 V, minimum charging voltage of 6V, and charging mode of CC/CV, with the lining and shell made of polyester. It will be understood by a person skilled in the art that other suitable choices of batteries could be used for the purpose as well. FIG. 2 shows device 200 incorporating power bank battery 230 having USB style plug 272 and plug 270. In some instances, the battery can be disposed on the mask itself, as shown in FIG. 3, wherein rejuvenating device 300 includes mask 320 having clothing pocket 325 configured to store battery 330.

The infrared radiation element can be embedded into the mask, between the layers of the mask. Alternatively, it can be disposed on the interior of the mask, and in some instances on the exterior of the mask. The infrared radiation element is configured to emit infrared radiation in the middle infrared range of 3-30 μm. In some instances, the infrared radiation element is configured to emit radiation in the range of 30-100 μm. Preferably, in the range of 6-10 μm, and most preferably at 8.5 μm. The infrared radiation element can include one or more strips having a plurality of infrared LEDs attached thereto. In some instances, it can incorporate one or more infrared lasers. The human immune system is enhanced by exposure to infrared radiation with the wavelength of 5 microns to 20 microns and the devices of the present invention are configured to be used to treat skin as well as to prevent diseases, including Covid-19 infections. According to the embodiments of the present invention, the mask incorporating an infrared radiation element can be any medical protective mask such as KN95 mask, or preferably a reusable respirator mask, or a surgical mask such as a 3-ply face mask, or a Kitsbow Wake Protech reusable face mask, or a reusable cotton mask, or any other reusable suitable protective masks. The infrared radiation element is positioned in close proximity to the skin of the user. In some instances, the distance between the skin of the user and the infrared radiation element is in the range from 1 mm to 10 mm. In a more preferred embodiment, the distance is in the range from 1 mm to 6 mm; and yet in a preferred embodiment, the distance is in the range from 1 mm to 3 mm. In some instances, the infrared radiation element can be in direct contact with the skin of the user.

According to one embodiment of the present invention, the infrared radiation element is an electrically operated heating pad, which can include a flexible black body radiant heat generating layer configured to radiate the energy from its surface at a pre-determined wavelength when the current is conducted therethrough, a pair of flexible electrically insulating and radiant energy permeable layers covering the opposite sides of the layer, a thermal insulation layer and a reflective layer. The flexible radiant heat generating layer can include a fiber glass mat impregnated with resistive material. In some instances, the infrared radiation element can be a heating pad radiating the infrared radiation form the heating coils by conduction. It can include a plurality of insulated electrical heating elements held within a sealed cloth envelop. It could further include a thermostat to control the amount of heat generated. This type of heating pads is resistance heated by the flow of electricity therethrough, which heat the surrounding insulated envelop. The technical result of the present invention is achieved, among other things, by the fact the infrared radiation element is positioned in closed proximity to the skin, unlike the designs of the prior art devices, wherein the infrared radiation element is positioned too far from the skin of a user to be an effective and efficient provider of a skin rejuvenating treatment.

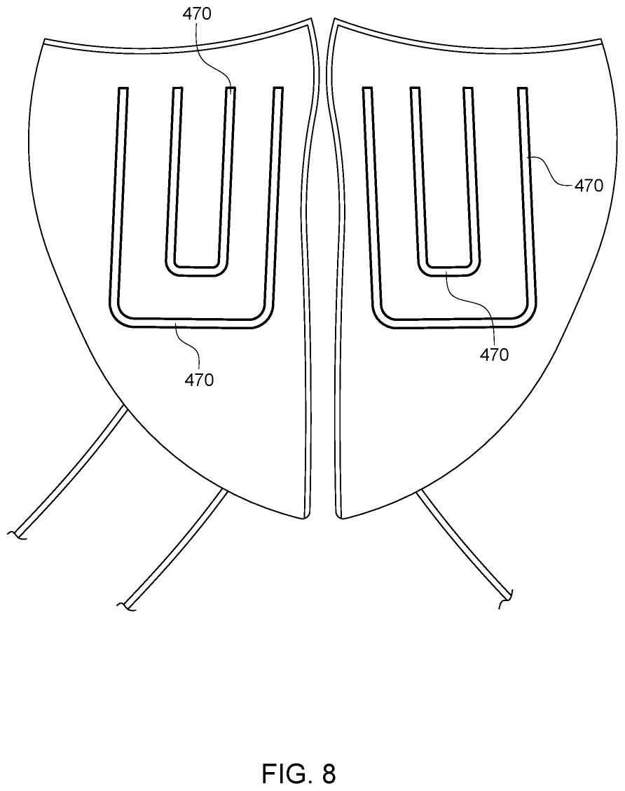

According to some embodiments of the present invention, the infrared radiation element can be made of carbon fiber. It was discovered that a carbon fiber infrared element can heat and treat the skin more efficiently in comparison with copper and other metals, because it has a very low coefficient of thermal expansion. The infrared radiation element can be incorporated into a mask in a pattern as illustrated in FIG. 8, for example, wherein infrared radiation element 470 forming a U-shaped pattern. According to other embodiments of the present invention, the infrared radiation element can form various patterns such as a circular, square, oval or any other suitable pattern. In some instances, the infrared radiation element can be incorporated into a mask as a combination of two or more patterns (e.g., oval and square or U-shaped and circular, etc.). According to some embodiments of the present invention, the infrared radiation element does not form a pattern but can consist of a plurality of separate infrared radiation elements. In some instances, the infrared radiation element can be a single element that is sized to partially fill the area of the gloves or the face mask.

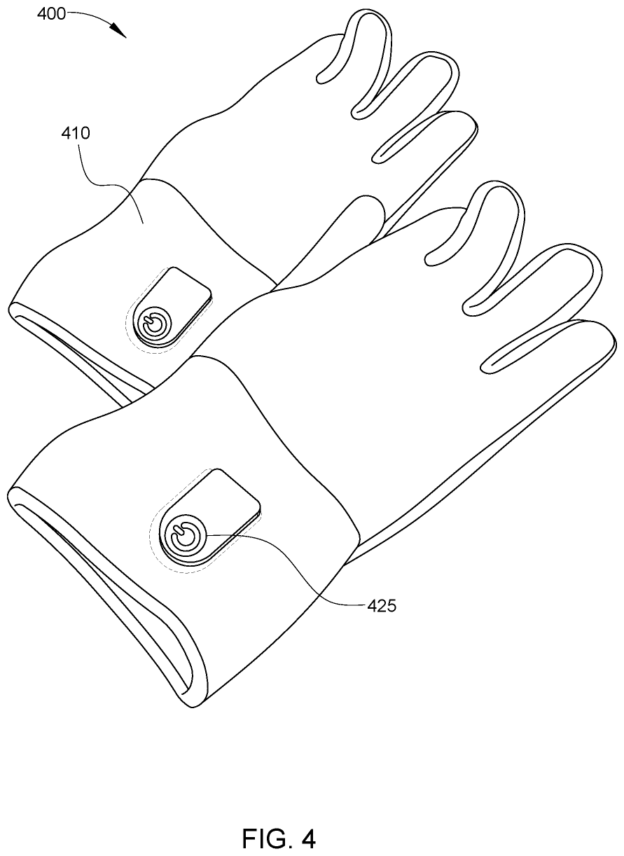

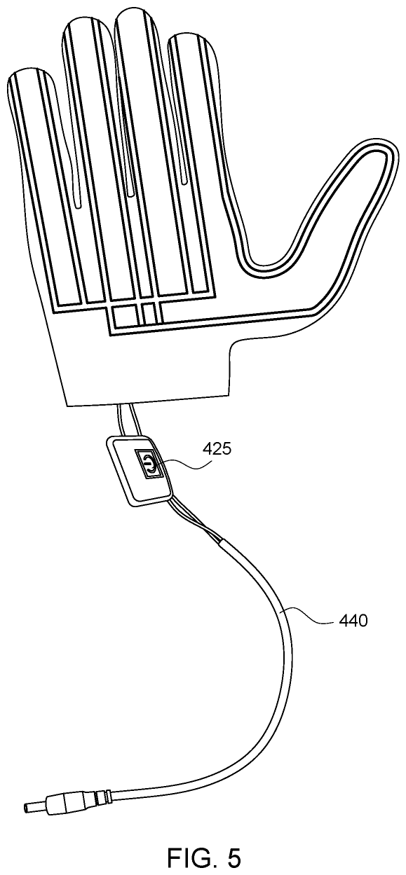

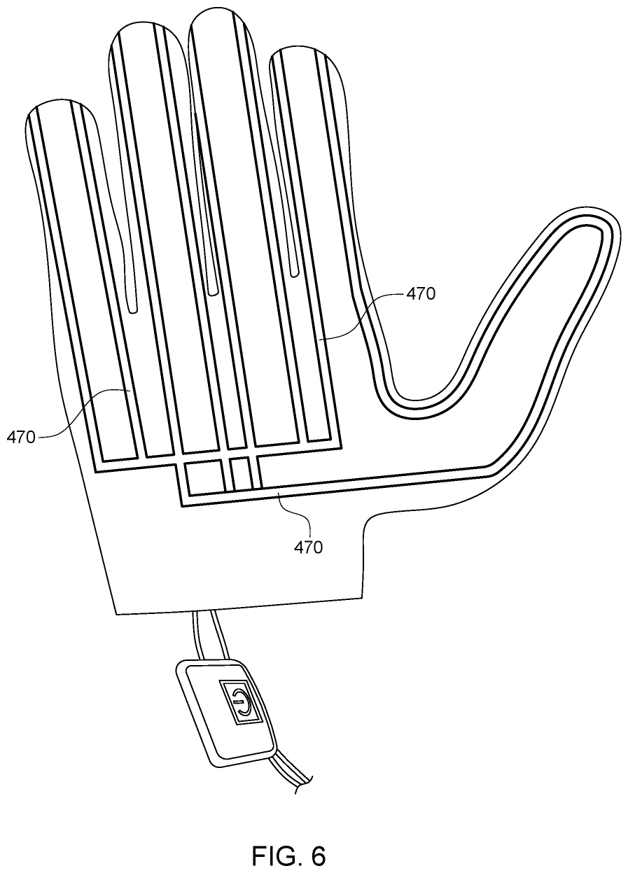

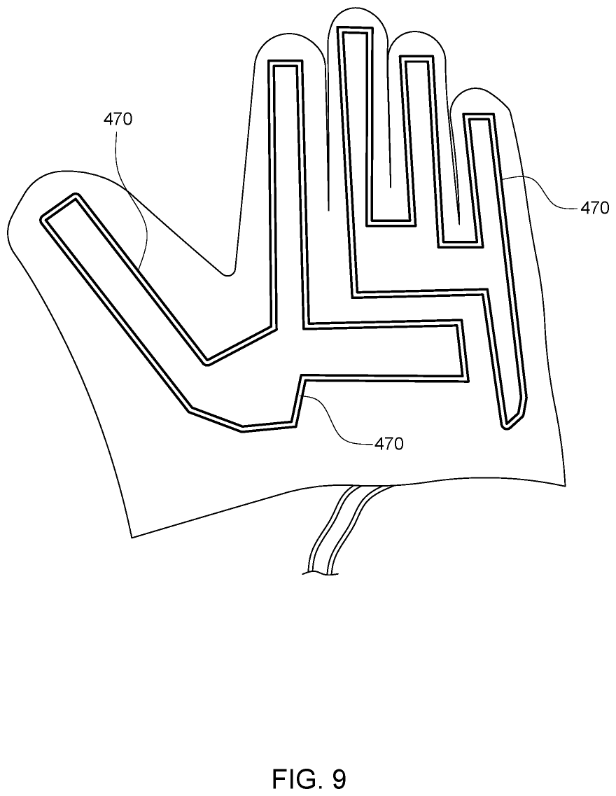

Referring now to FIG. 4, similarly to the treatment of face using the cloth face mask incorporating the infrared element as described above, the same principles can be applied for the treatment of hands in accordance with the embodiments of the present invention, wherein the infrared radiation element is incorporated into device 400, which includes a pair of gloves 410 with an infrared radiation element (not shown) embedded into the gloves and a power button 425, which can be placed outside of the gloves as shown in FIG. 5, with electrical wires 440 connected to a battery. Infrared heating element 470 can include a plurality of heating pad lines 470 as shown in FIG. 6, for example. The gloves as described above can be used alone for the purpose of skin rejuvenating treatment of hands. In some instances, the gloves can be used in conjunction with the face mask as a kit. The infrared radiation element incorporated into the gloves can form various patterns such as shown in FIG. 9 for example, wherein infrared radiation element 470 forming a pattern that substantially outlines the contour of a hand. In some instances, the pattern can be circular, or U-shaped, or oval or square or of any other suitable form, or a combination of two or more patterns. According to some embodiments of the present invention, the infrared radiation element does not form a pattern but can consist of a plurality of separate infrared radiation elements. In some instances, the infrared radiation element can be a single element that is sized to partially fill the area of the gloves or the face mask. In some instances, the distance between the skin of the user and the infrared radiation element is in the range from 1 mm to 10 mm. In a more preferred embodiment, the distance is in the range from 1 mm to 6 mm; and yet in a preferred embodiment, the distance is in the range from 1 mm to 3 mm. In some instances, the infrared radiation element can be in direct contact with the skin of the user.

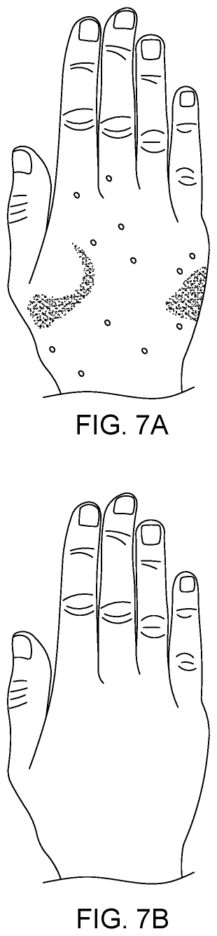

In order to achieve beneficial results, the devices of the present invention should be used for 15-20 minutes per day for at least 10 days. In some instances, the devices should be used for at least 20 days, and in some instances for 30 days, as further illustrated by FIG. 7A (a skin condition of hands before the treatment) and FIG. 7B (a skin condition of hands after the treatment), wherein the device of the present invention was used for at least 10 days.

The mask incorporating the infrared radiation element as disclosed above can be used as a stand-alone device for skin treatment of face. In some instances, the mask can be used in combination with gloves incorporating infrared radiation element as disclosed above as a kit for skin treatment of face and hands. The infrared radiation element can also be incorporated into various pieces of clothing such as bra, for example to treat various areas of skin, in a similar fashion as described above in relation to a face mask and gloves.

It will be understood that the system and method may be embodied in other specific forms without departing from the spirit or central characteristics thereof. The present examples and embodiments, therefore, are to be considered in all respects as illustrative and not restrictive, and the device and method is not to be limited to the details given herein.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

Although the invention is described herein with reference to specific embodiments, various modifications and changes can be made without departing from the scope of the present invention as set forth in the claims below. Accordingly, the specification and figures are to be regarded in an illustrative rather than a restrictive sense, and all such modifications are intended to be included within the scope of the present invention. Any benefits, advantages, or solutions to problems that are described herein with regard to specific embodiments are not intended to be construed as a critical, required, or essential feature or element of any or all the claims.

Unless stated otherwise, terms such as “first” and “second” are used to arbitrarily distinguish between the elements such terms describe. Thus, these terms are not necessarily intended to indicate temporal or other prioritization of such elements.

The foregoing detailed description is merely exemplary in nature and is not intended to limit the invention or application and uses of the invention. Furthermore, there is no intention to be bound by any expressed or implied theory presented in the preceding technical field, background, brief summary, or the following detailed description.

What is claimed is:

1. A wearable device for therapeutic skin treatment using infrared radiation, comprising:
a base member having an infrared radiation element coupled to the base member and a power source coupled to the base member and electrically connected to the infrared radiation element; wherein the infrared radiation element is positioned in close proximity to the skin of a user.

2. The device of claim 1, wherein the infrared radiation element is embedded into the base member between a first layer and a second layer.

3. The device of claim 1, wherein the infrared radiation element is disposed on the interior of the base member.

4. The device of claim 1, wherein the infrared radiation element is disposed on the exterior of the base member.

5. The device of claim 1, wherein the infrared radiation element is configured to emit infrared radiation in the middle infrared range of 3-30 μm.

6. The device of claim 1, wherein the infrared radiation element is configured to emit infrared radiation in the range of 30-100 μm.

7. The device of claim 1, wherein the infrared radiation element comprising one or more strips having a plurality of infrared light emitting diodes (LEDs) attached thereto.

8. The device of claim 1, wherein the infrared radiation element comprising one or more strips having a plurality of infrared lasers.

9. The device of claim 1, wherein the distance between the skin of the user and the infrared radiation element is in the range from 1 mm to 6 mm.

10. The device of claim 1, wherein the base member is shaped as a protective mask.

11. The device of claim 1, wherein the base member is shaped as a glove.

12. The device of claim 1, wherein the infrared radiation element is an electrically operated heating pad having a flexible black body radiant heat generating layer configured to radiate the energy from its surface at a pre-determined wavelength when the current is conducted therethrough, a pair of flexible electrically insulating and radiant energy permeable layers covering the opposite sides of the flexible black body radiant heat generation layer, a thermal insulation layer and a reflective layer.

13. The device of claim 12, wherein the flexible black body radiant heat generation layer further comprising a fiber glass mat impregnated with resistive material.

14. The device of claim 1, wherein the infrared radiation element is a heating pad radiating the infrared radiation from heating coils by conduction.

15. The device of claim 1, wherein the infrared radiation element is made of carbon fiber.

16. The device of claim 1, wherein the infrared radiation element is incorporated into the base member in a pattern.

17. The device of claim 1, wherein the infrared radiation element is in direct contact with the skin of the user.

18. A kit for therapeutic skin treatment using infrared radiation, comprising a first base member shaped as a mask and having a first infrared radiation element coupled to the first base member and at least one second base member shaped as a glove and having a second infrared radiation element coupled to the at least one second base member.

19. A method of using the device of claim 1, comprising the steps of:
activating the device by turning on the power source; and
wearing the device daily for 15-20 minutes for at least 10 days.

20. The method of claim 19, further comprising the step of wearing the device daily for 15-20 minutes for additional 10 days.
